(19) United States
(12) Patent Application Publication
(10) Pub. No.: US 2014/0336595 A1
(43) Pub. Date: Nov. 13, 2014

(54) PERIPHERAL ADMINISTRATION OF PROTEINS INCLUDING TGF-beta SUPERFAMILY MEMBERS FOR TREATMENT OF SYSTEMIC DISORDERS AND DISEASE

(71) Applicant: STRYKER CORPORATION, Kalamazoo, MI (US)

(72) Inventors: Mary Elizabeth Pecquet Goad, Andover, MA (US); Melissa M. Haskell, Uxbridge, MA (US); Denis Schrier, Hopkinton, MA (US); Susan A. Wood, Dedham, MA (US)

(73) Assignee: STRYKER CORPORATION, Kalamazoo, MI (US)

(21) Appl. No.: 14/186,545

(22) Filed: Feb. 21, 2014

Related U.S. Application Data
(63) Continuation of application No. 13/945,317, filed on Jul. 18, 2013.
Application No. 13/945,317 is a continuation of application No. 13/680,381, filed on Nov. 19, 2012, now abandoned.
Application No. 13/680,381 is a continuation of application No. 13/444,282, filed on Apr. 11, 2012, now abandoned.
Application No. 13/444,282 is a continuation of application No. 12/705,259, filed on Feb. 12, 2010, now abandoned.
(60) Provisional application No. 61/151,909, filed on Feb. 12, 2009.

(51) Int. Cl. A61M 5/00; A61K 9/00; A61K 38/18

(52) U.S. Cl. CPC A61M 5/00; A61K 38/1875; A61K 38/1841; A61K 9/0019
USPC 604/272; 514/8.8; 514/8.9

(57) ABSTRACT

The present invention is directed to methods and compositions for accomplishing systemic delivery of minimally-soluble bioactive agents such as, but not limited to, proteins of the TGF-β superfamily via a peripheral mode of administration. According to the invention, an exemplary bioactive agent is BMP-7. The invention further provides for minimally-invasive systemic treatment of skeletal disorders such as osteoporosis as well as minimally-invasive systemic treatment of injured or diseased non-mineralized tissues and organs such kidneys. Practice of the invention eliminates adverse side effects at the peripheral site of intravenous administration of the bioactive agent.

### CROSS-REFERENCE TO RELATED APPLICATIONS

This application claims priority to and the benefit of U.S. Provisional Patent Application No. 61/151,909, filed Feb. 12, 2009, the contents of which are incorporated by reference herein.

### BACKGROUND

Bone morphogenetic proteins (BMPs) belong to the superfamily of transforming growth factor β (TGF-β), and control a diverse set of cellular and developmental processes, such as pattern formation and tissue specification as well as promoting wound healing and repair processes in adult tissues. BMPs were initially isolated by their ability to induce bone and cartilage formation; however, their utility for other tissue and organ repair is now widely appreciated.

To date, a reliable means for non-local delivery of a clinically effective dose of a BMP—especially over a prolonged period of time, without repeated administration of the BMP—has eluded the skilled practitioner. In fact, effective delivery of most proteinaceous biologic agents generally remains an unanswered challenge. Despite progress in protein technologies and pharmaceutical chemistries, at least two problems continue to plague clinicians needing to provide therapeutic doses of a BMP(s) to patients.

First, the preferred mode of administration of most therapeutic agents is oral or by injection. However, oral administration is often inappropriate for macromolecular drugs such as proteins, as most of them are not absorbed intact by the gastrointestinal tract which can compromise the efficacy of a particular dosage regimen. Moreover, when a particular therapeutic protein or proteinaceous agent is administered by routine injection methods, frequent and multiple injections are required because these agents can have short pharmacokinetic half lives. Thus, the most popular and routine means of administering medications can pose a substantial physical burden on the patient and create significant administrative costs related to patient management.

Thus, there is a need for alternative modes of providing biologically active agents, especially macromolecules such as BMPs and other proteinaceous macromolecular biologics or drugs.

### SUMMARY OF THE INVENTION

The present invention is based on the discovery that an exemplary BMP, BMP-7, can be provided non-surgically and non-locally to mammals without injuring the vasculature. Practice of the present invention permits multiple and repeated intravascular peripheral administration of BMP-7 for systemic therapeutic purposes without the adverse effects heretofore observed. Typically, peripheral intravenous administration of BMPs using routine injection methods is accompanied (in rats, dogs, and monkeys, for example) by marked local effects including, but not limited to, edema, fibrosis, and formation of bone and/or cartilage in and around both the vessel and perivascular tissues at the site of the introduction of the needle and/or catheter. These undesirable local effects preclude injection of the protein into the same vessel more than once or twice, thereby necessitating use of many different peripheral vessels, leading to an accumulation of undesired effects in every vessel used for injection. In clinical practice, development of such adverse effects precludes administration more than once per week and, after 4 to 5 injections, eventually leads to an inability to inject the protein intravenously into any peripheral blood vessel.

The present invention exploits the discovery that, when intravascularly administering BMP-7 to a peripheral site, intima tissue integrity at the actual delivery site should be substantially uncompromised. That is, if at the actual delivery site, any of the protein leaks around the vessel, into the vessel wall, or into the surrounding tissues, then the undesired effects of edema, fibrosis, and formation of bone and/or cartilage in and around the vessel and/or perivascular tissues will occur. The distance between the site of administration or venipuncture and the site of delivery is optimally a distance that permits delivery of the therapeutic agent, for example, BMP-7, beyond the point of any trauma or damage to the vessel and associated tissues including the lumen of the vessel, the perivascular and subcutaneous tissues, thereby preventing leakage of the therapeutic agent into the surrounding tissues. The distance between the site of administration or venipuncture and the site of delivery depends on a variety of factors including the type of the vessel (e.g., artery or vein) the size of the vessel (large or small), and the anatomy of the vessel (e.g., presence of valves, branches etc.). Prevention of leakage is of particular importance in the administration of BMPs such as BMP-7 in order to prevent the particular side effects at the site of administration otherwise associated with BMP-7, namely cartilaginous or bony growths or nodules. Thus, practice of the present invention involves directing delivery of protein into a blood vessel downstream from or past a point (for example, at least about 1 to about 3 cm) of any trauma or damage (e.g., the physical injection or vascular puncture site; the administration site) to the vessel since such trauma or damage permits leakage of the protein into the tissues of the vessel or into surrounding tissues. When practiced as described herein, the invention enables protein delivery into a segment of blood vessel which is free of holes, tears, and/or trauma induced by the physical penetration of the blood vessel.

As contemplated herein, the invention involves placement of a catheter through a regular needle or a catheter introducer about 1 cm past, at least about 1 cm past, about 2 cm past or at least about 2 cm past and up to about 5 cm past, the venipuncture site, but in any case at least beyond any induced trauma to the vessel. For example, the catheter alone (without the needle or introducer) is first positioned past the immediate site of introduction into the vessel lumen and is then threaded preferably about 1 cm, at least about 1 to about 2 cm, at least 2 cm, more than 2 cm, more preferably about 2 to about 4 cm, and as far as up to about 40 cm, past the site of venipuncture and/or past any associated trauma. Only then is the protein actually delivered. The diameter (gauge) of the needle and catheter, as well as the length of catheter used, are dependent upon the species, age, sex, weight, and/or size of the patient. Any peripheral vessel can be used (e.g., veins or arteries in hands, arms, legs, feet and/or the jugular vein), but typically the preferred vessel is a vein in the subcutis of an extremity.

As is explained elsewhere herein, aspects of the invention further include a protein formulation suitable for minimally-invasive systemic delivery, including formulation parameters such as pH, excipients and/or concentration to name but a few, as well as the rate of administration of such formulations and effective dosages of the same accomplished via manipulation of formulation parameters and/or rates of administration.

Given the availability of preclinical evidence confirming that a number of systemic disease states could benefit from BMP therapy, the importance of the above-described minimally-invasive approach to systemic therapy can not be underestimated. In particular, metabolic bone diseases including mineralized as well as non-mineralized tissues affected thereby are of significant importance. Additionally, preclinical research confirms a number of systemic disease states for which BMP therapy can be beneficial including tissues and/or organs affected by diseases or disorders such as chronic and acute kidney disease, atherosclerosis, pulmonary fibrosis, obesity, diabetes, cancer, ocular scarring, liver fibrosis, inflammatory disorders and nervous system disorders.

In a first aspect, the present invention is directed to a composition comprising a biologic agent and a venipuncture apparatus for administering the agent to blood vessel. The venipuncture apparatus is adapted to deliver the agent to a region of the blood vessel which is trauma-free. In certain preferred embodiments, a biologic agent is a minimally soluble protein. In one embodiment, the proteinaceous biologic agent is a protein that is substantially insoluble at physiological pH. In one embodiment, the proteinaceous biologic agent is a member of the TGF-β superfamily of proteins. Another embodiment of the present invention provides for a proteinaceous biologic agent that is a member of the BMP subfamily of the TGF-β superfamily of proteins. In one embodiment of the present invention, the proteinaceous biologic agent is BMP-2, BMP-4, BMP-5, BMP-6, BMP-7, GDF-5, GDF-6, or GDF-7. In another aspect of the present invention, the proteinaceous biologic agent is BMP-7. The present invention also provides for a proteinaceous biologic agent that is sequence variant of any one of BMP-2, BMP-4, BMP-5, BMP-6, BMP-7, GDF-5, GDF-6, or GDF-7. In another aspect of the present invention, the proteinaceous biologic agent is a protein having at least about 50% amino acid sequence identity with a member of the BMP subfamily within the conserved C-terminal cysteine-rich domain.

In accordance with the present invention, a venipuncture apparatus is one which provides access to a subject's vasculature; the vasculature is preferably a peripheral blood vessel. In the case of the present invention, the venipuncture apparatus is adapted to deliverer the agent to a region of the subject's peripheral blood vessel which is trauma-free. As contemplated by the present invention, a preferred vascular access structure is implantable on the exterior or the interior of a subject's periphery. In certain embodiments, such a structure can operate to deliver a biologic agent into the subject's blood stream at a site remote from the actual implantation site, for example, a venipuncture or insertion site. In still other preferred embodiments, the venipuncture apparatus is equipped with a non-damaging distal end.

In one embodiment, the invention provides a method for treating a disease in a patient by systemically administering a bone morphogenetic protein to a patient in need thereof. The method includes the step of administering the bone morphogenetic protein to the patient at an administrative site via a vascular access structure. The administration site is peripheral and the bone morphogenetic protein is delivered to the patient a peripherally located delivery site at least 1 cm from the administration site. In a further embodiment, the delivery site is at least 2 cm, or at least 4 cm, or at least 5 cm from the delivery site. In another embodiment, the method further includes the step of implanting a vascular access structure at the peripheral administration site in the patient. For example, the peripheral administration site is a vein in a hand, a leg, a foot, an arm, or a head of the patient. In a further embodiment, the peripherally located delivery site is substantially edema free and substantially non-perturbed, while in another embodiment, the delivery site is venular-valve free. According to a further embodiment, the bone morphogenetic protein administered is BMP-7. According to a further embodiment, the bone morphogenetic protein administered at least three times in three separate administrations at the administration site.

In another embodiment, the invention provides a method for treating a disease in a patient by systemically administering a bone morphogenetic protein to a patient in need thereof. The method includes the step of administering the bone morphogenetic protein to the patient at an administrative site via a vascular access structure healed into the patient. According to this embodiment, the administration site is peripheral and the bone morphogenetic protein is delivered to the patient at a peripherally located delivery site about 2 cm from the administration site. For example, according to some embodiments, the peripheral administration site is a vein or artery in a foot, a hand, a head, an arm, or a leg or the patient. In other embodiments, the bone morphogenetic protein is BMP-7. According to some embodiments, the bone morphogenetic protein is administered to the patient multiple times at the site of administration. In a further embodiment, after multiple administrations, the patient does not exhibit side effects, i.e., nodule formation, bone formation or the like around the site of administration, incident with BMP administration.

In a currently preferred embodiment, a composition of the present invention suitable for ameliorating an injury or disease comprises a biologic agent selected from the group consisting of: a member of the TGF-β superfamily of proteins, a member of the BMP subfamily of the TGF-β superfamily of proteins, and a protein having at least about 50% amino acid sequence identity with a member of the BMP subfamily within the conserved C-terminal cysteine-rich domain; and, a venipuncture apparatus selected from the group consisting of a catheter, a needle, a catheter needle, an apparatus adapted to deliver said agent to a trauma-free region of the blood vessel, and a structure having functionally or structurally similar configurations thereto. As contemplated herein, the biologic agent is in an amount effective to ameliorate an injury or disease and wherein the venipuncture apparatus is adapted to deliver biologic agent to a trauma-free region of a selected blood vessel.

In another aspect, the present invention also provides for a formulation comprising a biologic agent in amount effective to ameliorate tissue injury or disease which is suitable for inclusion with the compositions described above. In one embodiment, the injury to be ameliorated is a mineralized or non-mineralized skeletal tissue injury. In another embodiment, the injury or disease to be ameliorated is metabolic bone disease, osteoarthritis, osteochondral disease, rheumatoid arthritis, osteoporosis, Paget's disease, periodontitis, dentinogenesis, chondral disease, trauma-induced and inflammation-induced cartilage degeneration, age-related cartilage degeneration, articular cartilage injuries and diseases, full thickness cartilage diseases, superficial cartilage defects, sequelae of systemic lupus erythematosis, sequelae of scleroderma, periodontal tissue regeneration, herniation and rupture of intervertebral discs, degenerative diseases of the intervertebral disc, osteocondrosis, or injuries and diseases of ligament, tendon, synovial capsule, synovial membrane and meniscal tissues. In another embodiment, the injury or disease to be ameliorated is liver disease, liver resection, hepatectomy, renal disease, chronic renal failure, central nervous system ischemia or trauma, neuropathy, motor neuron injury, dendritic cell deficiencies and abnormalities, Parkinson's disease, ophthalmic disease, ocular scarring, retinal scarring, or ulcerative diseases of the gastrointestinal tract. In yet another embodiment, the composition comprises biologic agent is in an amount effective to suppress tumor cell proliferation or promote tumor regression.

In yet another aspect, the present invention contemplates methods of systemic treatment using proteins such as but not limited to those of the TGF-β superfamily which are minimally invasive. As used herein, “systemic” means non-local. The skilled practitioner will understand that non-local can include a method whereby a protein or other bioactive agent is introduced to a subject at a single local site, such as but not limited to a peripheral percutaneous site, so as to effectuate treatment of the subject's whole body rather than just a single local site. “Systemic” can also mean that therapeutic blood levels of an administered therapeutic agent are present in the blood at a point in time. “Systemic” administration can also effectuate treatment of a site in a patient's body remote from the site of administration by providing therapeutic blood levels of an administered therapeutic agent. As used herein, “minimally-invasive” means non-invasive or non-open-field surgical methods. The skilled practitioner will understand that such methods can include procedures involving an incision(s) or implantation of a medical device(s).

In certain currently preferred embodiments, a method of treatment of an injured or diseased tissue comprises the step of providing to an administration site a composition comprising a biologic agent and a venipuncture apparatus for administering the agent to a blood vessel, whereupon the biologic agent is delivered in an amount effective to treat the injured or diseased tissue and the apparatus is adapted to deliver the agent to a trauma-free region of the blood vessel. Preferably, the physical administration site is remote from the actual site of delivery of the biologic agent. In a preferred embodiment, the administration site is a peripheral site.

In currently preferred embodiments, upon its delivery, the biologic agent disperses at a rate sufficient to provide a biologically effective dose at a site remote from the site of delivery. In a particularly preferred embodiment, the biologic agent disperses at a rate of at least 1 ml/min. In accordance with the present invention, the delivery site is substantially edema-free and/or substantially unperturbed and uncompromised. In yet another, the non-vascular tissue at, near or adjacent the delivery site is substantially free of biologic agent following delivery.

In another currently preferred embodiment of the invention, a method of treatment of an injured or diseased tissue comprises administering to an administration site a composition comprising a biologic agent and delivering to an intravenous delivery site the composition such that intima tissue integrity at the delivery site is substantially uncompromised. In this embodiment, the biologic agent disperses from the delivery site at a rate and in an amount effective to treat the injured or diseased tissue. In certain embodiments, the administration site and the delivery site are the same. In other embodiments, the delivery site is remote from the administration site. In one currently preferred embodiment, the site of delivery is about 1 cm downstream from the site of administration. In a particularly preferred embodiment, the delivery site is venular-valve-free. In another, the blood flow rate at the delivery site is sufficient to provide a biologically effective dose at a site remote from the site of delivery. In a particularly preferred embodiment, the biologic agent disperses at a rate of at least about 1 ml/min. In yet another, the delivering step is accomplished using an intravenous apparatus having a distal end with a non-damaging configuration.

In keeping with the teachings of the present invention, the currently preferred biologic agent is BMP-7 and injured or diseased non-mineralized tissue is the currently preferred object of treatment. Such injured or diseased tissue can be an organ. In a particularly preferred method of treatment, the biologic agent is bioavailable for at least about 0.5 hours, more preferably at least about 2 hours, at least about 8 hours; for about 1 day, preferably more than 1 day. And, an effective amount is about 10 microgram to about 1000 microgram of biologic agent, more preferably about 50 microgram to about 500 microgram, most preferably 100 microgram to about 300 microgram.

In another aspect, the present invention also provides for a formulation comprising a biologic agent in amount effective to ameliorate tissue injury or disease which is suitable for use with the methods described above.

In another aspect, the invention provides kits for use in systemically administering a bone morphogenetic protein to a patient in need thereof. In one embodiment, such a kit includes a bone morphogenetic protein and a peripheral vascular access structure for peripheral implantation in a patient. In an alternative embodiment, the kit further includes instructions for systemically administering the bone morphogenetic protein to the patient via peripheral administration. In a further embodiment, the instructions provide that a point of administration and a point of delivery of the bone morphogenetic protein should be greater than 1 cm apart for administration in humans. In a further embodiment, the bone morphogenetic protein is BMP-7. In yet a further embodiment, the bone morphogenetic protein is provided in a composition comprising a suitable pharmaceutical carrier.

In another embodiment, a kit includes a bone morphogenetic protein and instructions for systemically administering the bone morphogenetic protein to the patient via peripheral administration. In an another embodiment, the kit further includes a peripheral vascular access structure for peripheral implantation in a patient. In yet another embodiment, the bone morphogenetic protein is BMP-7.

The foregoing, and other features and advantages of the invention as well as the invention itself, will be more fully understood from the following figures, description, and claims.

### DETAILED DESCRIPTION

The present invention is based on the discovery that an exemplary bone morphogenetic protein (BMP), BMP-7, can be provided non-surgically and non-locally to mammals without adverse effects. Practice of the present invention permits multiple and repeated intravascular peripheral administration of such an exemplary protein for systemic therapeutic purposes without the adverse effects heretofore observed. Typically, peripheral intravenous administration of BMPs such as BMP-7 using routine injection methods is accompanied (in rats, dogs, and monkeys, for example) by marked local effects including, but not limited to, edema, fibrosis, and formation of bone and/or cartilage in and around both the vessel and perivascular tissues at the site of the introduction of the needle and/or catheter. These undesirable local effects preclude injection of the protein into the same vessel more than once or twice, thereby necessitating use of many different peripheral vessels, leading to an accumulation of undesired effects in every vessel used for injection. In clinical practice, development of such adverse effects precludes administration more than once per week and, after 4 to 5 injections, eventually leads to an inability to inject the protein intravenously into any peripheral blood vessel.

The present invention exploits the discovery that, when intravascularly administering an exemplary protein such as BMP-7 to a peripheral site, intima tissue integrity at the actual delivery site should be substantially uncompromised. That is, if at the actual delivery site, any of the protein leaks around the vessel, into the vessel wall, or into the surrounding tissues, then the undesired effects of edema, fibrosis, and formation of bone and/or cartilage in and around the vessel and/or perivascular tissues will occur. Thus, practice of the present invention involves directing delivery of protein into the blood downstream from or past a point (for example, at least about 1 to about 3 cm) of any trauma or damage (e.g., the physical injection or vascular puncture site; the administration site) to the vessel since such trauma or damage permits leakage of the protein into the tissues of the vessel or into surrounding tissues. When practiced as described herein, the invention enables protein delivery into a segment of blood vessel which is free of holes, tears, and/or trauma induced by the physical penetration of the blood vessel. As contemplated herein, the invention involves placement of a catheter through a regular needle or a catheter introducer at least about 1 cm past, and up to about 5 cm past, the venipuncture site, but in any case at least beyond any induced trauma to the vessel. For example, the catheter alone (without the needle or introducer) is first positioned past the immediate site of introduction into the vessel lumen and is then threaded preferably at least about 1 to about 2 cm, more preferably about 2 cm to about 4 cm, and as far as up to about 40 cm, past the site of venipuncture and/or past any associated trauma. Only then is the protein actually delivered. The diameter (gauge) of the needle and catheter, as well as the length of catheter used, are dependent upon the species, age, sex, weight, and/or size of the patient. Any peripheral vessel can be used (e.g., hands, arms, legs, feet and/or the jugular vein), but typically the preferred vessel is a vein in the subcutis of an extremity.

As is explained elsewhere herein, aspects of the invention further include a protein formulation suitable for minimally-invasive systemic delivery, including formulation parameters such as pH, excipients and/or concentration to name but a few, as well as the rate of administration of such formulations and effective dosages of the same accomplished via manipulation of formulation parameters and/or rates of administration.

Given the availability of preclinical evidence confirming that a number of systemic disease states could benefit from BMP therapy, the importance of the above-described minimally-invasive approach to systemic therapy can not be underestimated. In particular, metabolic bone diseases including mineralized as well as non-mineralized tissues affected thereby are of significant importance. Additionally, preclinical research confirms a number of systemic disease states for which BMP therapy can be beneficial including tissues and/or organs affected by diseases or disorders such as chronic and acute kidney disease, atherosclerosis, pulmonary fibrosis, obesity, diabetes, cancer, ocular scarring, liver fibrosis, inflammatory disorders and nervous system disorders.

As explained earlier, when an agent such as a TGF_{β} family member is injected directly into a peripheral blood vessel via a needle and syringe or using a standard intravenous temporary catheter (a catheter and needle delivery device), this is associated with marked local effects including but not limited to edema, fibrosis, and formation of bone and/o cartilage in and around the vessel and perivascular tissues at the site of the introduction of the needle and/or catheter (e.g., the venipuncture site). In contrast, directed delivery of the same protein via a peripheral catheter extending into the vessel's lumen for about 2 or more cm, for example, past the site of vessel puncture the typical undesired effects of edema, fibrosis, and formation of bone and/or cartilage in and around the vessel and/or perivascular tissues are absent or significantly diminished at the immediate site of the introduction of the needle and/or catheter. As will be understood by one of skill in the art, distance traversed is dependent upon the size of the species receiving the protein intravascularly; e.g., an entire rat may be about 8 cm in length head to tail whereas the upper forelimb of a dog may itself measure 8 cm.). A typical arm or hand blood vessel in an adult human has a straight length such that from the site of needle trauma at the site of administration, the tip of the catheter can be inserted at least 2 cm beyond the point of administration prior to delivery. In a further embodiment, a catheter can be introduced at least 2 cm beyond the distal tip of a fully inserted introducing needle prior to delivering the substance.

Moreover, as is explained elsewhere herein, aspects of the invention further include a protein formulation suitable for minimally-invasive systemic delivery, including formulation parameters such as pH, excipients and/or concentration to name but a few, as well as the rate of administration of such formulations and effective dosages of the same accomplished via manipulation of formulation parameters and/or rates of administration.

While current clinical applications of proteins such as BMPs, as well as other members of the TGF-β superfamily of tissue morphogens, are limited to local, surgically-invasive implantation for inducing local bone growth and repair, preclinical research confirms a number of systemic disease states for which BMP therapy can be beneficial. These include but are not limited to applications in metabolic bone diseases including mineralized as well as non-mineralized tissues affected thereby. Additionally, preclinical research confirms a number of systemic disease states for which BMP therapy can be beneficial including tissues and/or organs affected by diseases or disorders such as chronic and acute kidney disease, atherosclerosis, pulmonary fibrosis, obesity, diabetes, cancer, ocular scarring, liver fibrosis, inflammatory disorders and nervous system disorders. In accordance with the treatment of such diseases using the present invention, non-local administration of BMP-7 is now appreciated to be the optimal approach. However, the present invention further confirms that conventional, currently-employed methods of systemic administration, such as direct peripheral administration (e.g., via subcutaneous, intramuscular or intraperitoneal administration; further including intravenous administration using a syringe equipped with a traditional syringe needle) can have undesirable effects, including the formation of ectopic bone and/or fibrous tissue at the injection site and/or inducement of localized tissue trauma such as for example peripheral edema. As is explained elsewhere herein, the present invention relates to heretofore-undescribed methods for circumventing such undesirable effects and facilitate minimally-invasive systemic delivery of a biologic agent, especially a proteinaceous macromolecule such as but not limited to a BMP. It is further understood that minimally-invasive systemic delivery as contemplated herein does not include oral, parenteral or topical delivery.

### Biologic Agents Including Bone Morphogenetic Proteins

In brief, the present invention contemplates that a suitable biologic agent is proteinaceous. One suitable proteinaceous agent is a minimally soluble protein. That is, a preferred biologic agent is a protein that is substantially insoluble at physiological pH. For example, one exemplary proteinaceous biologic agent is a member of the TGF-β superfamily of proteins. The present invention further provides for a proteinaceous biologic agent that is a member of the BMP subfamily of the TGF-β superfamily of proteins. In certain embodiments of the present invention, the proteinaceous biologic agent is BMP-2, BMP-4, BMP-5, BMP-6, BMP-7, GDF-5, GDF-6, or GDF-7. In a preferred embodiment of the present invention, the proteinaceous biologic agent is BMP-7. The present invention also provides for a proteinaceous biologic agent that is sequence variant of any one of BMP-2, BMP-4, BMP-5, BMP-6, BMP-7, GDF-5, GDF-6, or GDF-7. In another embodiment of the present invention, the proteinaceous biologic agent is a protein having at least about 50% amino acid sequence identity with a member of the BMP subfamily within the conserved C-terminal cysteine-rich domain.

As stated above, BMPs are a preferred exemplary biologic agent for purposes of the present invention and belong to the TGF-β superfamily. The TGF-β superfamily proteins are cytokines characterized by six-conserved cysteine residues. The human genome contains about 42 open reading frames encoding TGF-β superfamily proteins. The TGF-β superfamily proteins can at least be divided into the BMP subfamily and the TGF-β subfamily biologic agents based on sequence similarity and the specific signaling pathways that they activate. The BMP subfamily includes, but is not limited to, BMP-2, BMP-3 (osteogenin), BMP-3b (GDF-10), BMP-4 (BMP-2b), BMP-5, BMP-6, BMP-7 (osteogenic protein-1 or OP-1), BMP-8 (OP-2), BMP-8B (OP-3), BMP-9 (GDF-2), BMP-10, BMP-11 (GDF-11), BMP-12 (GDF-7), BMP-13 (GDF-6, CDMP-2), BMP-15 (GDF-9), BMP-16, GDF-1, GDF-3, GDF-5 (CDMP-1, MP-52), and GDF-8 (myostatin). For purposes of the present invention, preferred superfamily proteins include BMP-2, -4, -5, -6 and -7 and GDF-5, -6, and -7, as well as MP-52. Particularly preferred proteins include BMP-2, BMP-7 and GDF-5, -6, and -7. A most preferred exemplary BMP is BMP-7. BMPs are also present in other animal species. Furthermore, there is allelic variation in BMP sequences among different members of the human population, and there is species variation among BMPs discovered and characterized to date.

The TGF-β subfamily includes, but is not limited to, TGFs (e.g., TGF-β1, TGF-β2, and TGF-β3), activins (e.g., activin A) and inhibins, macrophage inhibitory cytokine-1 (MIC-1), Mullerian inhibiting substance, anti-Mullerian hormone, and glial cell line derived neurotrophic factor (GDNF). As used herein, “TGF-β subfamily,” “TGF-βs,” “TGF-β ligands” and grammatical equivalents thereof refer to the TGF-β subfamily members, unless specifically indicated otherwise.

The TGF-β superfamily is in turn a subset of the cysteine knot Cytokine superfamily. Additional members of the cysteine knot cytokine superfamily include, but are not limited to, platelet derived growth factor (PDGF), vascular endothelial growth factor (VEGF), placenta growth factor (PIGF), noggin, neurotrophins (BDNF, NT3, NT4, and βNGF), gonadotropin, follitropin, lutropin, interleukin-17, and coagulogen.

Publications disclosing these sequences, as well as their chemical and physical properties, include: BMP-7 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)), BMP-2, BMP-4, (WO88/00205; Wozney et al. Science, 242, pp. 1528-1534 (1988)), BMP-5 and BMP-6, (Celeste et al., PNAS, 87, 9843-9847 (1991)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/U595/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/U594/00657) and WO96/14335 (PCT/U594/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)). The above publications are incorporated herein by reference.

As used herein, “TGF-β superfamily member” or “TGF-β superfamily protein,” means a protein known to those of ordinary skill in the art as a member of the Transforming Growth Factor-β (TGF-β) superfamily. Structurally, such proteins are homo or heterodimers expressed as large precursor polypeptide chains containing a hydrophobic signal sequence, an N-terminal pro region of several hundred amino acids, and a mature domain comprising a variable N-terminal region and a highly conserved C-terminal region containing approximately 100 amino acids with a characteristic cysteine motif having a conserved six or seven cysteine skeleton. These structurally-related proteins have been identified as being involved in a variety of developmental events.

The term “morphogenic protein” refers to a protein belonging to the TGF-β superfamily of proteins which has true morphogenic activity. For instance, such a protein is capable of inducing progenitor cells to proliferate and/or to initiate a cascade of events in a differentiation pathway that leads to the formation of cartilage, bone, tendon, ligament, neural or other types of differentiated tissue, depending on local environmental cues. Thus, morphogenic proteins useful in this invention can behave differently in different surroundings. In certain embodiments, a morphogenic protein of this invention can be a homodimer species or a heterodimer species.

The term “osteogenic protein (OP)” refers to a morphogenic protein that is also capable of inducing a progenitor cell to form cartilage and/or bone. The bone can be intramembranous bone or endochondral bone. Most osteogenic proteins are members of the BMP subfamily and are thus also BMPs. However, the converse can not be true. According to this invention, a BMP identified by DNA sequence homology or amino acid sequence identity must also have demonstrable osteogenic or chondrogenic activity in a functional bioassay to be an osteogenic protein. Appropriate bioassays are well known in the art; a particularly useful bioassay is the heterotopic bone formation assay (see, U.S. Pat. No. 5,011,691; U.S. Pat. No. 5,266,683, for example).

Structurally, BMPs are dimeric cysteine knot proteins. Each BMP monomer comprises multiple intramolecular disulfide bonds. An additional intermolecular disulfide bond mediates dimerization in most BMPs. BMPs may form homodimers. Some BMPs may form heterodimers. BMPs are expressed as pro-proteins comprising a long pro-domain, one or more cleavage sites, and a mature domain. The pro-domain is believed to aid in the correct folding and processing of BMPs. Furthermore, in some but not all BMPs, the pro-domain may noncovalently bind the mature domain and may act as an inhibitor (e.g., Thies et al. (2001) Growth Factors 18:251-259).

BMPs are naturally expressed as pro-proteins comprising a long pro-domain, one or more cleavage sites, and a mature domain. This pro-protein is then processed by the cellular machinery to yield a dimeric mature BMP molecule. The pro-domain is believed to aid in the correct folding and processing of BMPs. Furthermore, in some but not all BMPs, the pro-domain may noncovalently bind the mature domain and may act as a chaperone, as well as an inhibitor (e.g., Thies et. al. (2001) Growth Factors, 18:251-259).

As contemplated herein, the term “BMP” refers to a protein belonging to the BMP subfamily of the TGF-β superfamily of proteins defined on the basis of DNA homology and amino acid sequence identity. According to this invention, a protein belongs to the BMP subfamily when it has at least 50% amino acid sequence identity with a known BMP subfamily member within the conserved C-terminal cysteine-rich domain that characterizes the BMP subfamily. Members of the BMP subfamily can have less than 50% DNA or amino acid sequence identity overall. As used herein, the term “BMP” further refers to proteins which are amino acid sequence variants, domain-swapped variants, and truncations and active fragments of naturally occurring bone morphogenetic proteins, as well as heterodimeric proteins formed from two different monomeric BMP peptides, such as BMP-2/7; BMP-4/7: BMP-2/6; BMP-2/5; BMP-4/7; BMP-4/5; and BMP-4/6 heterodimers. Suitable BMP variants and heterodimers include those set forth in US 2006/0235204; WO 07/087053; WO 05/097825; WO 00/020607; WO 00/020591; WO 00/020449; WO 05/113585; WO 95/016034 and WO93/009229.

The terms “drug,” “medicament,” or “biologic agent”/“biologic agent” (i.e., biologically active agent) as used herein include without limitation biologically, physiologically or pharmacologically active substances that can act locally or systemically in the body. A biologic agent is a substance used for the treatment, prevention, diagnosis, cure or mitigation of disease or illness, a substance which affects the structure or function of the body, or pro-drugs, which become biologically active or more active after they reside in or contact a preferred physiological environment. Also, various forms of a biologic agent can be used. These include without limitation forms such as uncharged molecules, molecular complexes, salts, ethers, esters, amides, etc., which are biologically activated when injected into the body. Preferred biologic agents include, but are not limited to, proteins having therapeutic or prophylactic activity, including enzymes, growth factors, hormones, differentiation factors, cytokines, chemokines, and antibodies.

To those skilled in the art, any biologic agent that can be released in an aqueous environment can be utilized in the disclosed invention. In a preferred embodiment, the biologic agent is proteinaceous. In another preferred embodiment, the biologic agent is minimally soluble. In a more preferred embodiment, the biologic agent is substantially physiologically insoluble. In a further preferred embodiment, the biologic agent is substantially insoluble at physiological pH.

In another preferred embodiment, the biologic agent is one that can persist, after dosing, in vivo, with effectiveness for 1 hour, more preferably 24 hours, more preferably 48 hours, still more preferably one week, still more preferably one month, yet still more preferably several months. In a more particularly preferred embodiment, the biologic agent is a member of the TGF-β superfamily. In a still more particularly preferred embodiment, the biologic agent is selected from the group consisting of BMP-2, BMP-4, BMP-5, BMP-6, BMP-7, GDF-5, GDF-6, GDF-7, as well as any and all variants and homologues thereof. For instance, useful BMPs include those containing sequences, which are homologues or variants, that share at least 50%, preferably at least 60%, more preferably at least 70% and most preferably at least 85%, amino acid sequence identity with the C-terminal cysteine domain of BMP-2, BMP4, BMP-5, BMP-6, BMP-7, GDF-5, GDF-6, or GDF-7. As contemplated herein, preferred BMPs include biologically active variants of any such BMPs, including variants containing conservative amino acid substitutions. All that is required by the present invention is that these variants retain biological activity comparable to the native form. As used herein, the term “BMP related protein” or “BMP related proteins” means any one or all of the foregoing proteins.

Morphogenic proteins useful herein include any known naturally occurring native proteins, including allelic, phylogenetic counterparts and other variants thereof. These variants include forms having varying glycosylation patterns, varying N-termini, and active truncated or mutated forms of a native protein. Useful morphogenic proteins also include those that are biosynthetically produced (e.g., “muteins” or “mutant proteins”) and those that are new, morphogenically active members of the general morphogenic family of proteins.

### Modes of Administration and Delivery; Including Vascular Access Structures

Of particular importance, the present invention contemplates methods of systemic treatment using proteins, such as but not limited to those of the TGF-β superfamily, which are minimally invasive. As used herein, “systemic” means non-local. The skilled practitioner will understand that non-local can include a method whereby a protein or other bioactive agent is introduced to a subject at a single local site, such as but not limited to a peripheral percutaneous site, so as to effectuate treatment of the subject's whole body rather than just at the single local site. As used herein, “minimally-invasive” means non-invasive or non-open-field surgical methods. The skilled practitioner will understand that minimally-invasive methods can include procedures involving an incision(s) or implantation of a medical device(s).

As already stated, the present invention is based on the discovery that a minimally-soluble bioactive agent can be provided to a subject other than by conventional routes such as oral administration, peritoneal injection, or repetitive peripheral injections. That is, a minimally-soluble bioactive agent such as a protein can now be provided effectively via a systemic route without adverse effects and without surgical intervention.

For purposes of this invention, “delivery site” means the anatomical site at which the bioactive agent actually comes into direct contact with blood; whereas, “administration site” means the anatomical site at which the bioactive agent is physically first introduced to a recipient. An administration site can experiences trauma due to introduction of a needle and/or catheter at the site.

The invention exploits the discovery that certain specific physiological criteria are determinative in successful administration and delivery of a minimally-soluble bioactive agent such as a protein, including an exemplary protein such as BMP-7. In the first instance, practice of the invention requires that the intravenous site of actual delivery be substantially uncompromised. For example, the most preferred site is trauma-free; for example, edema-free.

The integrity of perivascular, vascular and/or vessel intima tissue at the most preferred site is intact. Indicia of intima integrity are the extent to which protein enters or leaks into the vascular, perivascular and/or nonvascular tissue at the delivery site; no leakage or penetration of the tissue is preferred. That is, according to the teachings of the present invention, vascular, perivascular and/or non-vascular tissue at, near or adjacent the delivery site should be substantially free of biologic agent following delivery. Each of the foregoing indicia is readily measurable by one of ordinary skill in the art using routine materials and methods.

Another of the criterion for optimal practice of the invention is that the preferred delivery site should be venular-valve-free; preferably, a most preferred delivery site should not be in close proximity to a venular valve. The foregoing parameter is readily discernable by one of ordinary skill in the art using routine materials and methods.

Yet another criterion for practicing the present invention relates to the actual location of the delivery site relative to the administration site. In accordance with the present invention, the proximity of the actual delivery site to the actual administration site and/or the site of venous puncture must be monitored. The invention is a process that will allow multiple (repeated) intravascular injections of the protein (such as a TGF_{β} family member including BMP-7 as a specific example) into patients for therapeutic purposes. The primary accomplishments of this process are to introduce (inject) the drug into a blood vessel past a point (for example, at least about 1 to about 3 cm) of any trauma or damage (vascular puncture site) to the vessel that would allow leakage of the drug into the tissues of that vessel or into surrounding tissues. Therefore, the process is one that enables injection into a blood vessel that has no holes, tears, trauma or that would allow for perivascular or paravascular leakage of the injected drug. In certain embodiments, this process allows placement of a catheter through a regular needle or catheter introducer about 1 to about 5 cm past the point of needle and catheter trauma to the vessel (again dependent on the size of the animal/patient). That is, the catheter alone (without the needle or introducer) is placed past the immediate site of vascular puncture or introduction into the vessel lumen and is threaded about 1 cm, at least about 2 to about 4 cm and up to about 40 cm past the site of the puncture in certain embodiments. For example, in a preferred embodiment, a catheter is inserted to a minimum distance of 2 cm beyond the site of administration, that is, the point of trauma to the vessel, and delivery of the protein occurs at that minimum distance of 2 cm from the point of administration. In another embodiment, the delivery site is at least 2 cm beyond the distal tip of a fully inserted introducing needle. The diameter (gauge) of the needle and catheter and the length of catheter used are dependent upon the species and the age, sex, and weight and size of the patient. Any peripheral vessel could be used (on the hands, arms, legs, or feet as well as the jugular vein) but the typical vessel would be a vein in the subcutis of an extremity.

As mentioned previously, methods of the invention permit multiple, i.e., repeated injections of an exemplary protein at the same administration site without the need to locate a new site. Multiple injections are permitted, because, administration according to the methods of the invention described herein substantially reduces or eliminates side effects previously described herein. According to methods of the invention, a protein may be administered to a patient at the same administration site at least two, at least three, at least four, at least five, at least six, at least seven, at least eight, at least nine, or at least ten times. For example, a protein may be administered to a patient at the same administration site once daily or twice daily, for example, for one week, two weeks, three weeks, or four weeks. In a further embodiment, a protein may be administered to a patient at the same administration site once weekly, for example, for 2 weeks, 3 weeks, or 4 weeks. In a further embodiment, a protein may be administered to a patient at the same administration site every other day, for example, for 2 weeks, 3 weeks, or 4 weeks. In a further embodiment, a protein may be administered to a patent at the same administration site twice weekly, for example, for 2 weeks, 3 weeks, or 4 weeks.

When practicing the present invention, it is preferable that the actual delivery site be situated at least about 2 cm, or about to about 6 cm downstream from the actual administration site and/or the site of venous puncture; at least about 4 to about 10 cm; at least about 10 cm; or at least about 15 cm. For example, this is readily accomplished using a catheter introduced past the end of the needle or a catheter introducer; or any of the preferred venipuncture apparatus identified elsewhere herein in accordance with the methods of the present invention. Of course, the skilled practitioner will appreciate that optimal distance downstream from the actual administration site will vary depending upon the size of the patient.

In some embodiments, the location of the actual delivery site is determined by injecting a benign fluid such as 0.9% sterile saline solution at the proposed delivery site to ensure no leakage occurs. However, according to some embodiments, it is an advantage of the invention that the delivery site is at least 2 cm beyond the administration site so that leakage can routinely be avoided without having to first test for leakage with a benign fluid. For example, in humans the delivery site is at least 2 cm beyond the administration site so that leakage can routinely be avoided. In a further embodiment, in humans the delivery site is around 3 to 5 cm beyond the administration site so that leakage can routinely be avoided.

Thus, in a currently preferred embodiment of the invention, a method of treatment of an injured or diseased tissue comprises administering to an administration site a composition comprising a biologic agent and delivering to an intravenous delivery site the composition such that intima tissue integrity at the delivery site is substantially uncompromised. In this embodiment, the biologic agent disperses from the delivery site at a rate and in an amount effective to treat the injured or diseased tissue. In a particularly preferred embodiment, the delivery site is venular-valve-free. In another, the blood flow rate at the delivery site is sufficient to provide a biologically effective dose at a site remote from the site of delivery. In yet another, the delivering step is accomplished using an intravenous apparatus having a distal end with a non-damaging configuration.

In a related aspect, the present invention is directed to a composition comprising a biologic agent and a venipuncture apparatus or related apparatus. As contemplated herein, a venipuncture apparatus is any device or apparatus which can be used to provide an effective amount of biologic agent in accordance with the present invention. A preferred apparatus is one which does not disrupt or minimally disrupts intima tissue at the site of delivery. In accordance with the present invention, a venipuncture apparatus is a device which provides access to a subject's peripheral vasculature. As contemplated by the present invention, a venipuncture structure is implantable on the exterior or the interior of a subject. In certain embodiments, such an apparatus can operate to deliver a biologic agent into the subject's peripheral blood stream at a site remote from the implantation site.

The skilled practitioner will be familiar with such suitable venipuncture apparatus generally. Apparatuses contemplated herein include, for example, PICC catheters not introduced for their entire length but rather only for a length sufficient to extend past a site of vascular trauma while still providing a delivery site in a peripheral vessel.

In certain currently preferred embodiments, a method of treatment of an injured or diseased tissue comprises the step of providing to an administration site a composition comprising a biologic agent and a venipuncture apparatus, whereupon the biologic agent is delivered peripherally in an amount effective to treat systemically the injured or diseased tissue. As explained elsewhere herein, the physical administration site is peripheral and remote from the actual site of peripheral delivery of the biologic agent.

In keeping with the teachings of the present invention, the currently preferred biologic agent is BMP-7 and injured or diseased non-mineralized tissue is the currently preferred object of treatment. Such injured or diseased tissue can be an organ. In a particularly preferred method of treatment, the biologic agent is bioavailable for at least about 0.5 hours, more preferably at least about 2 hours, at least about 8 hours; for about 1 day, preferably more than 1 day. And, an effective amount is about 10 microgram to about 1000 microgram of biologic agent, more preferably about 50 microgram to about 500 microgram, most preferably about 100 microgram to about 300 microgram.

According to another aspect, the invention includes further methods of administration based on the insight described herein that intima tissue integrity at the delivery site must be substantially uncompromised for BMP, such as BMP-7, or any other protein disclosed herein, to be successfully administered without causing side effects as described herein. For example, according to the invention, the method includes administering BMP to a patient via a healed-in catheter. As a result of the catheter being healed-in, the tissues at the venipuncture site or administration site are not prone to edema and leakage of any administered agent into the surrounding tissue. For example, a healed-in catheter is maintained in a patient for several days such that the trauma at the venipuncture site has the opportunity to heal. For example, according to one embodiment of the invention, a healed-in catheter is a permanent catheter or port. According to the invention, the term “heal” or “healed” suggests that tissue integrity is substantially uncompromised and/or that the venipuncture site is substantially free of tissue damage. In other words, the term “heal” does not require complete repair of the injured or compromised site, although a tissue that is “healed” may be completely repaired or uncompromised. The term “heal” or “healed,” in one embodiment, suggests that damaged or diseased tissue has been substantially replaced with new tissue growth, which may include scar tissue.

According to one embodiment, BMP, such as BMP-7, is administered to a patient via a healed-in catheter in a peripheral vessel and the delivery site is less than 1 cm, about 1 cm, about 2 cm or more than 2 cm from the site of administration. According to another embodiment, the invention includes a method for administering a BMP to a patient. The method includes the step of introducing a catheter into a peripheral vein of a patient at an administration site, permitting the administration site to heal with the catheter in place such that the administration site is substantially uncompromised, and administering BMP to a patient via said catheter, wherein the delivery site is less than 1 cm, or about 1 cm, or about 2 cm or more than 2 cm from the site of administration of the site of administration.

The skilled artisan will appreciate that the treatment and administration methods of the present invention can be modified or varied to optimize treatment of an individual in view of numerous factors including, but not limited to, the indication, the pathology of the disease, and the physical characteristics of the individual.

### Therapeutic Interventions

As explained above, the invention also provides methods of treatment using a composition of the present invention containing any biologic agent, or formulation thereof, in an amount effective to ameliorate and/or prevent any known or potential condition for which the biologic agent is efficacious. As used herein “an effective amount” means an amount of a biologic agent that is effective to treat a condition in a living organism to which it is administered. For example, the BMP formulations of the invention can be used to treat patients suffering from disease or injury of connective tissues, such as bone and cartilage. Additionally, as described below, the BMP formulations of the invention can be used to treat diseases or injuries of other tissues.

In one embodiment of the invention, the injury to be ameliorated is a mineralized or non-mineralized skeletal tissue injury. In another embodiment, the injury or disease to be ameliorated is metabolic bone disease, osteoarthritis, osteochondral disease, rheumatoid arthritis, osteoporosis, Paget's disease, periodontitis, dentinogenesis, chondral disease, trauma-induced and inflammation-induced cartilage degeneration, age-related cartilage degeneration, articular cartilage injuries and diseases, full thickness cartilage diseases, superficial cartilage defects, sequelae of systemic lupus erythematosis, sequelae of scleroderma, periodontal tissue regeneration, herniation and rupture of intervertebral discs, degenerative diseases of the intervertebral disc, osteocondrosis, or injuries and diseases of ligament, tendon, synovial capsule, synovial membrane and meniscal tissues. In another embodiment, the injury or disease to be ameliorated is liver disease, liver resection, hepatectomy, renal disease, chronic renal failure, central nervous system ischemia or trauma, neuropathy, motor neuron injury, dendritic cell deficiencies and abnormalities, Parkinson's disease, ophthalmic disease, ocular scarring, retinal scarring, or ulcerative diseases of the gastrointestinal tract.

BMPs are capable of inducing the developmental cascade of bone morphogenesis and tissue morphogenesis for a variety of tissues in mammals different from bone or cartilage. This morphogenic activity includes the ability to induce proliferation and differentiation of progenitor cells, and the ability to support and maintain the differentiated phenotype through the progression of events that results in the formation of bone, cartilage, non-mineralized skeletal or connective tissues, and other adult tissues.

For example, BMPs can be used for treatment to prevent loss of and/or increase bone mass in metabolic bone diseases. General methods for treatment to prevent loss of and/or increase bone mass in metabolic bone diseases using osteogenic proteins are disclosed in U.S. Pat. No. 5,674,844, the disclosures of which are hereby incorporated by reference. BMPs of the present invention can be used for periodontal tissue regeneration. General methods for periodontal tissue regeneration using osteogenic proteins are disclosed in U.S. Pat. No. 5,733,878, the disclosures of which are hereby incorporated by reference. BMPs can be used for liver regeneration. General methods for liver regeneration using osteogenic proteins are disclosed in U.S. Pat. No. 5,849,686, the disclosures of which are hereby incorporated by reference. BMPs can be used for treatment of chronic renal failure. General methods for treatment of chronic renal failure using osteogenic proteins are disclosed in U.S. Pat. No. 6,861,404, the disclosures of which are hereby incorporated by reference. BMPs can be used for enhancing functional recovery following central nervous system ischemia or trauma. General methods for enhancing functional recovery following central nervous system ischemia or trauma using osteogenic proteins are disclosed in U.S. Pat. No. 6,407,060, the disclosures of which are hereby incorporated by reference. BMPs can be used for inducing dendritic growth. General methods for inducing dendritic growth using osteogenic proteins are disclosed in U.S. Pat. No. 6,949,505, the disclosures of which are hereby incorporated by reference. BMPs can be used for inducing neural cell adhesion. General methods for inducing neural cell adhesion using osteogenic proteins are disclosed in U.S. Pat. No. 6,800,603, the disclosures of which are hereby incorporated by reference. BMPs can be used for treatment and prevention of Parkinson's disease. General methods for treatment and prevention of Parkinson's disease using osteogenic proteins are disclosed in U.S. Pat. No. 6,506,729, the disclosures of which are hereby incorporated by reference.

Additionally, BMPs can be used to repair diseased or damaged mammalian tissue. The existing tissue at the locus, whether diseased or damaged, provides the appropriate matrix to allow the proliferation and tissue-specific differentiation of progenitor cells. In addition, a damaged or diseased tissue locus, particularly one that has been further assaulted by surgical means, provides a morphogenically permissive environment.

BMPs also can be used to prevent or substantially inhibit scar tissue formation following an injury. It can induce tissue morphogenesis at the locus, preventing the aggregation of migrating fibroblasts into non-differentiated connective tissue. For example, BMPs can be used for protein-induced morphogenesis of substantially injured liver tissue following a partial hepatectomy.

As another example, BMPs can also be used to induce dentinogenesis. To date, the unpredictable response of dental pulp tissue to injury is a basic clinical problem in dentistry. As yet another example, BMPs can induce regenerative effects on central nervous system (CNS) repair can be assessed using a rat brain stab model.

In the case of skeletal disorders, a number of factors can cause or contribute to cartilage degeneration in mammals, including trauma and inflammatory disease. Damage to cells resulting from the effects of inflammatory response has been implicated as the cause of reduced cartilage function or loss of cartilage function in diseases of the joints (e.g., rheumatoid arthritis (RA) and osteoarthritis (OA)). In addition, autoimmune diseases such as systemic lupus erythematosis (SLE) and scleroderma can also be characterized by a degradation of connective tissue. In the case of some cartilage degenerative diseases such as osteoarthritis (OA), the mechanisms that turn the normal aging of articular cartilage into the pathological OA process are currently unknown. Each of the foregoing diseases can be effectively treated with the materials and methods of the present invention.

### Formulations

Biologic agents, and especially BMPs, of the present invention can be formulated for administration to a mammal, preferably a human, in need thereof as part of a pharmaceutical composition. In a particularly preferred embodiment, the biologic agent is BMP-7. A currently preferred embodiment of the present invention comprises a BMP formulation comprising trehalose, preferably trehalose in a lactate buffer, most preferably BMP-7 in a buffer of 10 mM lactate comprising 9% trehalose. It is within the skill in the art to practice the aforementioned embodiments of the present invention, as well as any and all variants and modifications of the present invention that the skilled artisan would recognize provide effective dosing of the biologic agent in vivo.

Still further, the biologic agent of the present invention can be administered to the mammal in need thereof either alone or in combination with another substance known to have a beneficial effect on tissue morphogenesis. Examples of such substances (herein, cofactors) include without limitation substances that promote tissue repair and regeneration and/or inhibit inflammation. Examples of useful cofactors for stimulating bone tissue growth in osteoporotic individuals, for example, include but are not limited to, vitamin D₃, calcitonin, prostaglandins, parathyroid hormone, dexamethasone, estrogen and IGF-I or IGF-II. Useful cofactors for nerve tissue repair and regeneration can include, but are not limited to, nerve growth factors. Other useful cofactors include symptom-alleviating cofactors, including, but not limited to, antiseptics, antibiotics, antiviral and antifungal agents, analgesics and anesthetics.

As will be appreciated by those skilled in the art, the concentration of the compounds described in a therapeutic composition will vary depending upon a number of factors, including without limitation the dosage of the drug to be administered, the chemical characteristics (e.g., hydrophobicity) of the compounds employed, and the route of administration. The preferred dosage of drug to be administered also is likely to depend on variables including, but not limited to, the type and extent of a disease, tissue loss or defect, the overall health status of the particular patient, the relative biological efficacy of the compound selected, the formulation of the compound, the presence and types of excipients in the formulation, and the route of administration. The therapeutic molecules of the present invention may be provided to an individual where typical doses range from about 10 ng/kg to about 1 g/kg of body weight per day; with a preferred dose range being from about 0.1 mg/kg to 100 mg/kg of body weight, and with a more particularly preferred dosage range of 10-1000 μg/dose. The skilled clinician would appreciate that the effective doses of the present invention can be modified in light of numerous factors including, but not limited to, the indication, the pathology of the disease, and the physical characteristics of the individual. It is also clearly within the skill in the art to vary, modify, or optimize doses in view of any or all of the aforementioned factors.

Pursuant to the parameters and conditions of certain embodiments of the invention, the availability of the biologic agent can be controlled. In particular, the rate and extent of availability of the biologic agent from a formulation can be controlled by variation of properties such as but not limited to polymer type and molecular weight, use of a rate modifying agent, use of plasticizers and leachable agents and the concentrations and kinds of thermoplastic polymer and biologic agent.

Rate modifying agents, plasticizers and leachable agents can be included to manage the rate of release of biologic agent and the pliability of a matrix in which it is optionally contained. The rate modifying agent can increase or retard the rate of release depending upon the nature of the rate modifying agent incorporated into a matrix. Known plasticizers as well as organic compounds that are suitable for secondary pseudobonding in polymer systems are acceptable as rate modifying agents and also as pliability modifiers and leaching agents.

Generally these agents are esters of mono, di and tricarboxylic acids, diols and polyols, polyethers, non-ionic surfactants, fatty acids, fatty acid esters, oils such as vegetable oils, and the like. The concentrations of such agents within the matrix can range in amount up to 60 wt % relative to the total weight of the matrix, preferably up to 30 wt % and more preferably up to 15 wt %. Generally, these rate modifying agents, leaching agents, plasticizers and pliability modifiers and their application are described in U.S. Pat. Nos. 5,702,716 and 5,447,725, the disclosures of which are incorporated herein by reference with the proviso that the polymers to be used are biocompatible and/or biodegradable. The skilled artisan would appreciate that the present invention comprises any and all agents within the art that can increase the solubilization rate of the biologic agent or the degradation rate or erosion rate of any carrier for the biologic agent. Hence, other agents amenable to the practice of the present invention include, but are not limited to, co-localized pH modifying agents and tonicity modifiers. In a particularly preferred embodiment, the composition of the present invention comprises a co-localized pH modifying agent or tonicity modifier provided in a concentration or quantity that substantially increases the solubilization rate of the biologic agent. In another preferred embodiment, the composition of the present invention comprises a co-localized pH modifying agent or tonicity modifier provided in a concentration or quantity that substantially increases the degradation rate or erosion rate of the carrier. The skilled artisan would appreciate that the rate modifying agents, leaching agents, plasticizers, pliability modifiers, pH modifying agents, and tonicity modifiers of the present invention can be substituted, modified, varied in nature or concentration, and optimized in view of numerous factors, including, but not limited to, the desired release rate, the nature of the carrier (if any), the indication, the pathology of the disease, and the physical characteristics of the individual.

Formulations of biologic agents of this invention can further include one or more excipients. Examples of excipients are described in the Handbook of Pharmaceutical Excipients, published jointly by the American Pharmaceutical Association and the Pharmaceutical Society of Great Britain. Excipients that can be employed in the making and use of the formulations and pharmaceutical compositions of the present invention include, but are not limited to; acidifying agents, such as, acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid; alcohol denaturants, such as, denatonium benzoate, methyl isobutyl ketone, sucrose octacetate; alkalizing agents, such as, strong ammonia solution, ammonium carbonate, diethanolamine, diisopropanolamine, potassium hydroxide, sodium bicarbonate, sodium borate, sodium carbonate, sodium hydroxide, trolamine; antifoaming agents, such as, dimethicone, simethicone; antimicrobial preservatives, such as, benzalkonium chloride, benzalkonium chloride solution, benzelthonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol; antioxidants, such as, ascorbic acid, acorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipients; buffering agents, such as, acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate; chelating agents, such as, edetate disodium, ethylenediaminetetraacetic acid and salts, edetic acid; coating agents, such as, sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein; colors, such as, caramel, red, yellow, black or blends, ferric oxide; complexing agents, such as, ethylenediaminetetraacetic acid and salts (EDTA), edetic acid, gentisic acid ethanolmaide, oxyquinoline sulfate; dessicants, such as, calcium chloride, calcium sulfate, silicon dioxide; emulsifying and/or solubilizing agents, such as, acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, trolamine, emulsifying wax; filtering aids, such as, powdered cellulose, purified siliceous earth; glidants and/or anticaking agents, such as, calcium silicate, magnesium silicate, colloidal silicon dioxide, talc; humectants, such as, glycerin, hexylene glycol, propylene glycol, sorbitol; plasticizers, such as, castor oil, diacetylated monoglycerides, diethyl phthalate, glycerin, mono- and di-acetylated monoglycerides, polyethylene glycol, propylene glycol, triacetin, triethyl citrate; polymer membranes, such as, cellulose acetate; solvents, such as, acetone, acetic acid, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water; sorbents, such as, powdered cellulose, charcoal, purified siliceous earth, and carbon dioxide sorbents; stiffening agents, such as, hydrogenated castor oil, cetostearyl alcohol, cetyl alcohol, cetyl esters wax, hard fat, paraffin, polyethylene excipient, stearyl alcohol, emulsifying wax, white wax, yellow wax; suspending and/or viscosity-increasing agents, such as, acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum; and wetting and/or solubilizing agents, such as, benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol.

### Bioactive Co-agents

The present invention also contemplates “bioactive co-agents” that can be co-administered with the biologic agent compositions of the present invention include, but are not limited to, anabolic agents, antacids, anti-asthmatic agents, anti-cholesterolemic and anti-lipid agents, anti-coagulants, anti-convulsants, anti-diarrheals, anti-emetics, anti-infective agents including, for example, antibacterial and antimicrobial agents, anti-inflammatory agents, anti-manic agents, antimetabolite agents, anti-nauseants, anti-neoplastic agents, anti-bone resorption agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

More particularly, the bioactive co-agents preferred for co-administration with the present invention include, but are not limited to, androgen inhibitors, polysaccharides, growth factors, hormones, bisphosphonates, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainaminde, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines. Other representative bioactive co-agents that can be co-administered with the present invention include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, β-adrenergic blocking agents, nutritional agents, and the benzophenanthridine alkaloids. The bioactive co-agent may further be a substance capable of acting as a stimulant, sedative, hypnotic, analgesic, anticonvulsant, and the like.

The bioactive co-agent may also be a substance, or metabolic precursor thereof, which is capable of promoting growth and survival of cells and tissues, or augmenting the activity of functioning cells, as for example, blood cells, neurons, muscle, bone marrow, bone cells and tissues, and the like. For example, bioactive co-agents that may be co-administered include without limitation a nerve growth promoting substance, as for example, a ganglioside, phosphatidylserine, a nerve growth factor, brain-derived neurotrophic factor. The bioactive co-agent may also be a growth factor for soft or fibrous connective tissue as, for example, a fibroblast growth factor, an epidermal growth factor, an endothelial cell growth factor, a platelet derived growth factor, an insulin-like growth factor, a periodontal ligament cell growth factor, to name but a few.

### EXAMPLES

### 1. Minimally-Invasive Delivery of an Exemplary BMP via Peripheral Venous Delivery

(a) Peripheral Venous Delivery of BMP-7 in Dogs

The purpose of this study was to determine a method to safely deliver intravenous (IV) BMP-7 into peripheral veins of beagle dogs. This study had a pilot segment (2 adult female beagles) and Phases I and II (2 adult female beagles). The objective of the pilot portion of the study was to determine the optimal catheter system and gauge to allow repeated administration in a peripheral vessel. In Phase I of the study, dogs were dosed IV in a peripheral vein once per week for 4 injections; each injection administered 100 μg/kg BMP-7. Dogs were given an equal volume of vehicle alone in the contra lateral peripheral vessel. During Phase II of the study, the dosing frequency of 100 μg/kg BMP-7 was three times per week for four weeks.

In the Pilot Segment, one dog had two small, firm subcutaneous nodules on the left forelimb at the administration site (site of needle and catheter introduction) at approximately nine days post injection. This is likely due to perivascular injection of BMP-7 after unsuccessful catheterization attempts. The nodules resolved over approximately 8 weeks. The second pilot dog had a small region of subcutaneous thickening over the left dorsal forelimb at the administration site at approximately 9 days post injection, also likely due to perivascular injection of BMP-7 after unsuccessful catheterization attempts.

In Phase I and Phase II, dogs were injected using Peripherally Inserted Central Catheters (PICC). Catheters ranged from 26 to 20 gauge, and were successfully advanced in the vessels inserted from 4 cm and up to 40 cm from the insertion site. In Phase I, both dogs were successfully dosed once per week for 4 weeks with no abnormal clinical signs. In Phase II, with a BMP-7 dosing frequency of 3 times per week for 4 weeks, the PICC systems ranged from 28 to 20 gauge and were advanced from 12 to 30 cm past the insertion site depending on catheter used. Both dogs were successfully dosed three times per week for 4 weeks with no abnormal clinical signs. In Phase I and II, dogs had no clinical signs considered to be associated with IV administration of BMP-7.

(b) Evaluation of Peripheral Vein Irritation Associated with BMP-7 Administered Intravenously to Adult Female Beagle Dog

The purpose of this study was to determine a method to safely deliver intravenous (IV) BMP-7 into peripheral veins of beagle dogs. The objective of this study was to determine the optimal catheter system and gauge to allow repeated administration in a peripheral vessel, and to examine injection sites histologically. Dogs were dosed IV in a peripheral vein three times per week for two weeks, a total of 6 injections; each injection administered 100 μg/kg BMP-7. The distance from the distal tip of the fully inserted catheter needle to the tip of the catheter passed into the blood vessel lumen was at least 2 cm. Dogs were given an equal volume of vehicle alone in the contra-lateral peripheral vessel. The dogs injected with BMP-7 in which the PICC line was appropriately used and placed had no evidence of inflammation, fibrosis, or any other reactions. The dog (#7) that had an anatomical variation (a branching and small cephalic vein) with noted gross findings at necropsy and clinically had focal fibrosis (grossly, a small 1 cm diameter nodule) at the injection site due to puncture of the vein with the PICC line and administration of the BMP-7 extravascularly.

(c) Evaluation of Intravenous Toxicity and Local Tolerance in Beagle Dogs

The purpose of this study was to assess the toxicity and local tolerance of BMP-7 after repeated intravenous (IV) injections twice per week for a total of 8 injections into peripheral veins of female beagle dogs. The distance from the distal tip of the fully inserted catheter needle to the tip of the catheter passed into the blood vessel lumen was at least 2 cm. Three dogs per group for a total of 12 dogs were injected IV into a peripheral vein twice per week with vehicle control (Group 1, 5 mM lactose and 9% trehalose), or BMP-7 (Group 2-0.1 mg/kg BMP-7, Group 3-0.3 mg/kg BMP-7, and Group 4-1.0 mg/kg BMP-7).

Pre-dose (before IV injections) and termination (at euthanasia, ±1 days after the last IV injection) hematology, clinical chemistry, and coagulation profiles were analyzed for differences compared with baseline, control animals, and reference and historical ranges. Necropsy findings, organ weights, and microscopic findings at the injection sites (and gross findings) were analyzed comparing treated animals with control animals. All parameters evaluated, except for the microscopic findings, were within the range of normal for all animals at all time points.

Microscopically, all vessels used for drug administration via the PICC lines had no findings except in one animal. These findings were seen in only in this one dog as a single focus of bone in one Group 4 (1 mg/kg BMP-7) female that had a clinically observed penetration of the vessel by the catheter tip.

Under the conditions of this study, treatment of adult female beagle dogs with 8 IV injections (twice per week for 4 weeks) into a peripheral vein was associated with adverse effects (local bone formation) only in the single animal that had trauma and penetration of the vessel by the catheter.

Thus, these studies indicate that the practice of the present invention accomplishes peripheral administration of BMP-7 and similar biologic agents without local undesired effects and that the present invention will allow the clinical use of BMP-7 and similar biologic agents as a systemic therapeutic agent. Induction of the undesired local injection site effects would preclude the use of BMP-7 and similar biologic agents as intravenously administered drugs.

(d) Peripheral Venous Delivery of BMP-7 in Primates

Primate subjects will be administered a preparation of BMP-7 using a preferred venipuncture apparatus as contemplated herein; suitable control subjects will receive a protein-free preparation. Up to 10 mg/ml BMP-7 will be administered using a catheter introduced less than about 2 cm versus more than 2 cm past the site of needle-induced and/or catheter-induced trauma to the blood vessel. BMP-7 will be administered up to 3 times per week for up to 4 weeks. At least 4 subjects will be studied. Patients will be found to have no adverse side affects such as nodule formation or osteogenesis at the site of administration in patients having a delivery site more than 2 cm past the needle-induced or catheter-induced trauma while subjects receiving administration at less than about 2 cm are expected to demonstrate side affects such as nodule formation or osteogenesis at and around the site of administration.

### 2. Bone Morphogenetic Protein-7 as Therapy for an Adult Female Cat with Renal Insufficiency

An approximately 14 year old 2.5 kg female spayed cat with a history of renal insufficiency was administered 100 μg/kg BMP-7 intravenously via 28 gauge PICC catheter into the cephalic vein approximately once per month for four months. The PICC line was advanced at least 2 and up to 8 cm into the vein past the site of the needle introduction for delivery.

Blood was collected before each dose for serum chemistry analysis. Urine was collected before the first dose and after the second dose for urine specific gravity and urine protein/creatinine ratio. Clinical signs including appetite, attitude, urination volume, and coat quality as well as any observations at the injection sites were monitored by the owners. Creatinine (Cr) decreased to a more normal level by 13.6%. Blood urea nitrogen (BUN) showed no significant changes throughout the study. Urine specific gravity and urine protein/creatinine ratio were normal throughout the study.

Clinical signs before BMP-7 administration included increased urine volume, lethargy, unkempt (greasy) haircoat, poor appetite, and muscle wasting. After 4 doses of BMP-7, the urine volume had decreased to normal, the haircoat was markedly improved. The cat was noted to be more energetic and interactive. The animal had increased appetite and decreased muscle wasting; the cat had gained approximately 1 kg. The injection sites (the right and left cephalic veins) had no findings and were grossly normal. Based on the results of this study, BMP-7 may have potential to improve clinical signs in cats with mild renal insufficiency, especially when systemically administered through a peripheral vessel according to the methods of the invention.

### 3. Systemic Uses for Minimally-Invasive Peripheral Delivery of an Exemplary BMP in Humans

(a) Osteoporosis

A population of human patients with a confirmed clinical diagnosis of osteoporosis will be administered BMP-7 through a peripheral vessel in accordance with the methods of the present invention. In particular, a peripherally placed catheter is used to administer an i.v. dose of 0.01-3.0 μg/kg of BMP-7 once weekly and the BMP-7 is delivered at a point at least 2 cm from the point of administration and beyond any trauma caused by catheter or needle insertion into the vessel. It is expected that such treatment will modulate the disease to a statistically significant extent in the treated patient population and that there will be an absence of side effects incident with BMP-7 administration around the site of administration.

(b) Metabolic Bone Disease

A population of human patients with a confirmed clinical diagnosis of metabolic bone disease will be administered BMP-7 in accordance with the methods of the present invention. In particular, a peripherally placed catheter is used to administer an i.v. dose of 0.01-3.0 μg/kg of BMP-7 once weekly and the BMP-7 is delivered at a point at least 2 cm from the point of administration and beyond any trauma caused by catheter or needle insertion into the vessel. It is expected that such treatment will modulate the disease to a statistically significant extent in the treated patient population and that there will be an absence of side effects incident with BMP-7 administration around the site of administration.

(c) Fibrosis Including Hepatic, Pulmonary, Cardiac and Renal Manifestations

Populations of human patients with a confirmed clinical diagnosis of fibrosis including each of hepatic, pulmonary, cardiac and renal fibrosis will be administered BMP-7 in accordance with the methods of the present invention. In particular, a peripherally placed catheter is used to administer an i.v. dose of 0.01-3.0 μg/kg of BMP-7 once weekly and the BMP-7 is delivered at a point at least 2 cm from the point of administration and beyond any trauma caused by catheter or needle insertion into the vessel. It is expected that such treatment will modulate the disease in each treated population to a statistically significant extent and that there will be an absence of side effects incident with BMP-7 administration around the site of administration.

(d) Nerve and Spinal Cord Injuries

Populations of human patients with a confirmed clinical diagnosis of each of nerve and spinal cord injury will be administered BMP-7 in accordance with the methods of the present invention. In particular, a peripherally placed catheter is used to administer an i.v. dose of 0.01-3.0 μg/kg of BMP-7 once weekly and the BMP-7 is delivered at a point at least 2 cm from the point of administration and beyond any trauma caused by catheter or needle insertion into the vessel. It is expected that such treatment will modulate the disease in each treated population to a statistically significant and that there will be an absence of side effects incident with BMP-7 administration around the site of administration.

(e) Tumor Metastasis

A population of human patients with a confirmed clinical diagnosis of tumor metastasis will be administered BMP-7 in accordance with the methods of the present invention. In particular, a peripherally placed catheter is used to administer an i.v. dose of 0.01-3.0 μg/kg of BMP-7 once weekly and the BMP-7 is delivered at a point at least 2 cm from the point of administration and beyond any trauma caused by catheter or needle insertion into the vessel. It is expected that such treatment will modulate the disease to a statistically significant extent in the treated patient population and that there will be an absence of side effects incident with BMP-7 administration around the site of administration.

### EQUIVALENTS

The invention may be embodied in other specific forms without departing from the spirit or essential characteristics thereof. The present embodiments are therefore to be considered illustrative and not restrictive, the scope of the invention being indicated by the appended claims rather than by the foregoing description, and all changes which come within the meaning and range of equivalency of the claims are therefore intended to be embraced therein.

1. A method for treating a disease in a patient by systemically administering a bone morphogenetic protein to a patient in need thereof, the method comprising the step of:
administering the bone morphogenetic protein to the patient at an administrative site via a vascular access structure, wherein the administration site is peripheral and the bone morphogenetic protein is delivered to the patient at a peripherally located delivery site at least 1 cm from the administration site.

2. The method of claim 1, further comprising the step of implanting a vascular access structure at the peripheral administration site in the patient.

3. The method of claim 1, wherein the peripheral administration site is a vein in a hand, a leg, a foot, an arm or a head of the patient.

4. The method of claim 1, wherein the bone morphogenetic protein is BMP-7.

5. The method of claim 1, wherein the peripherally located delivery site is substantially edema free and substantially non-perturbed.

6. The method of claim 1, wherein the bone morphogenetic protein is administered multiple times to the patient via the administration site to the delivery site.

7. (canceled)

8. The method of claim 1, wherein the peripherally located delivery site is at least 2 cm, at least 4 cm, or at least 5 cm from the administration site.

9-21. (canceled)

22. A method of treatment of an injured or diseased tissue, the method comprising the step of:
administering to a peripheral administration site a composition comprising a biologic agent, and
delivering to a peripheral intravenous delivery site the composition such that intima tissue integrity at the delivery site is substantially uncompromised
whereupon the biologic agent is in an amount effective to treat the injured or diseased tissue.

23. The method of claim 22, wherein the administration site and the delivery site are the same.

24. The method of claim 22, whereupon the delivering step is accomplished using an intravenous apparatus having a distal end with a non-damaging configuration.

25. The method of claim 22, wherein the site of delivery is about 1 cm downstream from the site of administration.

26-36. (canceled)

37. The composition of claim 48, wherein the biologic agent is selected from the group consisting of GDF-5, GDF-6 and GDF-7.

38. The composition of claim 48, wherein the biologic agent is BMP-7.

39-41. (canceled)

42. The composition of claim 48, wherein the biologic agent is in an amount effective to ameliorate skeletal tissue injury or disease selected from the group consisting of: metabolic bone disease, osteoarthritis, osteochondral disease, rheumatoid arthritis, osteoporosis, Paget's disease, periodontitis, and dentinogenesis.

43. The composition of claim 48, wherein the biologic agent is in an amount effective to ameliorate non-mineralized skeletal tissue injury or disease selected from the group consisting of: osteoarthritis, osteochondral disease, chondral disease, rheumatoid arthritis, trauma-induced and inflammation-induced cartilage degeneration, age-related cartilage degeneration, articular cartilage injuries and diseases, full thickness cartilage defects, superficial cartilage defects, sequelae of systemic lupus erythematosis, sequelae of scleroderma, periodontal tissue regeneration, herniation and rupture of intervertebral discs, degenerative diseases of the intervertebral disc, osteocondrosis, and injuries and diseases of ligament, tendon, synovial capsule, synovial membrane and meniscal tissues.

44. The composition of claim 48, wherein the biologic agent is in an amount effective to ameliorate tissue injury selected from the group consisting of: trauma-induced and inflammation-induced cartilage degeneration, articular cartilage injuries, full thickness cartilage defects, superficial cartilage defects, herniation and rupture of intervertebral discs, degeneration of intervertebral discs due to an injury(s), and injuries of ligament, tendon, synovial capsule, synovial membrane and meniscal tissues.

45. The composition of claim 48, wherein the biologic agent is in an amount effective to ameliorate injury or disease of a tissue selected from the group consisting of: liver disease, liver resection, hepatectomy, renal disease, chronic renal failure, central nervous system ischemia or trauma, neuropathy, motor neuron injury, spinal cord injury, dendritic cell deficiencies and abnormalities, Parkinson's disease, ophthalmic disease, ocular scarring, retinal scarring, and ulcerative diseases of the gastrointestinal tract.

46. The composition of claim 48, wherein the biologic agent is in an amount effective to ameliorate injury or disease of a tissue selected from the group consisting of:
chronic and acute kidney disease, atherosclerosis, pulmonary fibrosis, cardiac fibrosis, renal fibrosis, obesity, diabetes, cancer, ocular scarring, liver fibrosis, inflammatory disorders and nervous system disorders.

47. (canceled)

48. A composition suitable for ameliorating an injury or disease comprising:
a biologic agent selected from the group consisting of: a member of the TGF-β superfamily of proteins, a member of the BMP subfamily of the TGF-β superfamily of proteins, and a protein having at least about 50% amino acid sequence identity with a member of the BMP subfamily within the conserved C-terminal cysteine-rich domain, wherein said biologic agent is in an amount effective to ameliorate an injury or disease; and,
a venipuncture apparatus for administering said agent to a blood vessel, the apparatus selected from the group consisting of: catheter, needle, catheter needle, catheter introducer, PICC line, and a structural equivalent of any one of the foregoing apparatus; wherein the apparatus is adapted to deliver said agent to a trauma-free region of the blood vessel.

49-57. (canceled)
